(19) United States
(12) Patent Application Publication
(10) Pub. No.: US 2011/0046660 A1
(43) Pub. Date: Feb. 24, 2011

(54) Endoscopic Forceps With Removable Handle

(75) Inventors: Baber Syed, Palo Alto, CA (US); Charles R. Brynelsen, Menlo Park, CA (US); Mitchell Lirtzman, Lafayette, LA (US); Ken Wong, Saratoga, CA (US)

Correspondence Address: IntraPace & Townsend & Townsend & Crew LLP, Two Embarcadero Center, Eighth Floor, San Francisco, CA, 94111-3834, US

(73) Assignee: IntraPace, Inc., Mountain View, CA (US)

(21) Appl. No.: 12/705,181

(22) Filed: Feb. 12, 2010

Related U.S. Application Data
(60) Provisional application No. 61/152,605, filed on Feb. 13, 2009.

(51) Int. Cl. A61B 17/28; A61M 29/00

(52) U.S. Cl. 606/206; 606/192

(57) ABSTRACT

An exemplary forceps system for use within a tool channel of an endoscope comprises an elongated body extending from a proximal end and a distal end having one or more internal lumens. An actuator is slidably positioned within a first lumen, and actuatable jaws are removeably coupled to a first end of the actuator near the distal end. A handle is removeably coupleable to the proximal end of the body, the removable handle having a forceps actuator operatively engageable with a second end of the actuator so as to control the actuatable jaws when the handle is coupled to the body. A single or double balloon closure device having an inflatable anchor on a peritoneal side and a narrow inflatable portion, is also provided. The narrow portion may be shaped to follow the shape of the transgastric cut. The balloon closure device may deliver medication speed up the healing process and may contain a biocompatible sealant that may be dispersed over the incision site and/or used to keep the anchor on the peritoneal side inflated.

### CROSS-REFERENCES TO RELATED APPLICATIONS

The present application claims the benefit under 35 USC 119(e) of U.S. Provisional Application No. 61/152,605 filed Feb. 13, 2009; the full disclosure of which is incorporated herein by reference in its entirety.

### STATEMENT AS TO RIGHTS TO INVENTIONS MADE UNDER FEDERALLY SPONSORED RESEARCH AND DEVELOPMENT

Not Applicable

### BACKGROUND OF THE INVENTION

The embodiments are related generally to medical devices, and more particularly to devices and methods useful in minimally invasive procedures, such as natural orifice translumenal endoscopic surgery (NOTES).

During minimally invasive surgeries, surgical tools are introduced into the body to carry out the desired treatments at a target location in the body. Minimally invasive procedures are desirable because such procedures can reduce pain and provide relatively quick recovery times as compared with conventional open medical procedures. Many minimally invasive procedures are performed with an endoscope, with the surgical tools being positioned within one or more tool or accessory channels in the endoscope. Such procedures permit a physician to position, manipulate, and view medical instruments and accessories inside the patient through a small access opening in the patient's body, such as insertion of medical instruments and accessories through a natural body orifice to a treatment region. Many of these procedures employ the use of a flexible endoscope during the procedure. Flexible endoscopes often have a flexible, steerable articulating section near the distal end that can be controlled by the user by utilizing controls at the proximal end. Minimally invasive therapeutic procedures to treat diseased tissue by introducing medical instruments to a tissue treatment region through a natural opening of the patient are known as Natural Orifice Translumenal Endoscopic Surgery (NOTES).

Some flexible endoscopes are relatively small (1 mm to 3 mm in diameter), and may have no integral tool or accessory channel. Other endoscopes have one or more tool or accessory channels having a diameter ranging from 2.0 to 6.0 mm for the purpose of introducing and removing medical devices and other accessory devices to perform the treatment within the patient. As a result, the accessory devices used by a physician can be limited in size by the diameter of the accessory channel of the scope used.

One drawback of using a tool or accessory in the endoscope channel is that when the endoscope is removed, the tool or accessory must also be removed with it. In some procedures, particularly procedures involving multiple operations such as endoscopic suturing of the gastric wall, it may be necessary to leave the tool or accessory in place while removing the endoscope.

It would be desirable to provide an endoscopic tool that can remain in place when the endoscope is removed. It also desirable to provide improved methods of using such a tool. And it would further be desirable to provide a simple closure device to close transgastric tracts or ports after performing NOTES.

### BRIEF SUMMARY OF THE INVENTION

In a first aspect, embodiments of the present invention provide a method comprising advancing an endoscope into an internal surgical site via a natural orifice (i.e. transgastric, transvaginal, or transanal), advancing jaws and a first end of an elongate flexible body of a forceps through a tool channel of the endoscope into the site, grasping a tissue with the forceps, removing a proximal handle from a second end of the elongate body of the forceps, retracting the endoscope out of the site over the elongate body while the forceps grasps the tissue, and replacing the handle on the forceps while the forceps grasps the tissue.

In another aspect, embodiments of the present invention provide a forceps system for use with an endoscope. The endoscopic system comprises an elongated body extending from a proximal end and a distal end having one or more internal lumens, an actuator slidably positioned within a first lumen, actuatable jaws removeably coupled to a first end of the actuator near the distal end and a handle removeably coupleable to the proximal end of the body, the removable handle having a forceps actuator operatively engageable with a second end of the actuator so as to control the actuatable jaws when the handle is coupled to the body.

In many embodiments, the forceps actuator is configured to move the actuatable jaws from a first position to a second position.

In many embodiments, the forceps actuator is configured to rotate the actuatable jaws.

In many embodiments, the actuatable jaws may be removed and replaced by another actuatable device, including at least one of: a snare, magnetic tool, a biopsy cup, a hook, or other suitable actuatable device.

In many embodiments, the forceps may be configured to make an electrical connection with an RF device to deliver RF energy at the distal actuatable device.

In many embodiments, the endoscopic system further comprises an actuator wire locking mechanism configured to lock the actuator wire within the first lumen. Locking the actuator wire also locks the actuatable device.

In many embodiments, one of the lumens is a guide wire lumen.

In many embodiments, the body and actuatable device are configured to slide within a tool or accessory channel of the endoscope.

In another aspect, the invention provides a method for resection of an appendix using natural orifice translumenal endoscopic surgery (NOTES). The method comprises creating a first port from a patient's stomach into the peritoneal cavity, advancing an endoscope orally into the stomach, through the first port into the peritoneal cavity, advancing a forceps with a removable handle through a tool channel of the endoscope into the peritoneal cavity, grasping the appendix at the base with the forceps and locking the forceps, removing the handle from the forceps, retracting the endoscope out of the mouth, leaving the forceps in place, replacing the handle on the forceps, creating a second port from the stomach into a peritoneal cavity, advancing the endoscope orally into the stomach, through the second port into the peritoneal cavity, advancing an endoscopic snare with electrocautery connection through the tool channel of the endoscope, placing the snare around the appendix, advancing an endoscopic grasper in a second tool channel and grasping the appendix, resecting the appendix with an electrocautery machine coupled to the snare, and removing the appendix while withdrawing the endoscope and grasper.

In many embodiments, the method further comprises manipulating the forceps to assist in placing the snare around the appendix.

In many embodiments, the method further comprises placing endoscopic clips around the base of the appendix prior to resection.

In many embodiments, the method further comprises closing the first and second ports using appropriate means.

In many embodiments, the method further comprises creating a first port and/or second port is done with an RF catheter.

In another aspect, the invention provides a closure device for temporarily closing a transgastric tract. The closure device comprises a catheter having a proximal end and a distal end, an inflation lumen within the catheter, and an inflatable balloon removeably coupled to the distal end, the balloon having a pressure valve in fluid communication with the inflation lumen such that the balloon remains inflated once uncoupled from the catheter, the balloon being sized to temporarily close the transgastric tract when inflated.

In many embodiments, the balloon has an antibiotic coating.

In many embodiments, the balloon is made of a material that allows it to shrink in size as the transgastric tract closes.

In many embodiments, the balloon is made of biodegradable material to promote natural passage through the gastric lumen as the healing progresses.

In many embodiments, the balloon is made of silicon or polyurethane.

In many embodiments, the invention comprises a single or double balloon closure device.

In many embodiments, the balloon closure device comprises an inflatable anchor on a peritoneal side.

In many embodiments, the balloon closure device may comprise a narrow inflatable portion, shaped to follow the shape of the transgastric cut.

In many embodiments, the balloon closure device may deliver medication of speed up the healing process.

In many embodiments, the balloon closure device may contain a biocompatible sealant that may be dispersed over the incision site and/or used to keep the anchor on the peritoneal side inflated.

In another aspect, the invention provides a method of closing a transgastric tract. The method comprises advancing a closure device to the transgastric tract, positioning of an inflatable balloon on a first end of the closure device across the transgastric tract, and inflating the balloon to seal the transgastric tract.

In many embodiments, the method further comprises uncoupling the inflated balloon from the closure device and withdrawing the closure device.

In many embodiments, advancing the closure device to the transgastric tract comprises positioning an endoscope proximate the transgastric tract and advancing the closure device through a tool channel of the endoscope.

In many embodiments, the method further comprises removing the balloon once the transgastric tract has healed.

In many embodiments, the method further comprises a device that is designed to deflate and naturally pass through the gastric lumen as the wound site heals.

In another aspect, the invention provides a transluminal crossing device comprising an elongated flexible body extending from a proximal end to a distal end. A tissue penetrating tip is disposed at the distal end so as to form a penetration in a wall of a body lumen. An expandable structure is disposed proximally of the tip, and the expandable structure has a small-profile configuration suitable for advancement of the expandable structure into the penetration. The expandable structure is expandable from the small profile configuration to a large-profile configuration, with that expansion being suitable for expanding the penetration when the wall surrounds the expandable structure.

In many embodiments, the radially expandable structure comprises a mechanism having a plurality of arms. Expansion of the mechanism comprises deploying the arms radially from along the body. Alternative embodiments may make use of a radially expandable structure comprising a balloon coupled to an inflation lumen of the body. Regardless, the expandable structure may also include a plurality of radially oriented blades disposed so that the blades radially incise tissue of the wall during the expansion. The wall will typically comprise a stomach wall, and the blades may inhibit or limit tearing of muscle or other tissue of the wall. The expandable structure may expand the penetration radially at least in part via dilation, with or without such blades.

### BRIEF DESCRIPTION OF THE DRAWINGS

FIG. 1A shows one embodiment of a forceps system with a removable handle.

FIG. 1B shows an alternative embodiment of a distal jaw for use in a forceps system with a removable handle.

FIGS. 2A-2D show a forceps system with a removable handle used in a Natural Orifice Translumenal Endoscopic Surgery (NOTES) to resect an appendix.

FIGS. 3A-3B show one embodiment of a closure device compatible for use with natural orifice translumenal endoscopic surgery (NOTES) to close a tract or port once the surgery or procedure is done.

FIG. 4 shows using the closure device of FIGS. 3A-3B to close a transgastric tract or port.

FIGS. 5A-5D show two embodiments of double balloon closure devices.

FIGS. 6A-6D show two embodiments of single balloon closure devices.

FIGS. 6E-6G show alternative embodiments of single and double balloon closure devices.

FIG. 7 shows one embodiment of a closure device catheter for use with a balloon closure device.

FIGS. 8A-8C show two embodiments of a Balloon Translumenal Crossing Device.

FIGS. 9A-9B show one embodiment of a Mechanical Translumenal Crossing Device.

### DETAILED DESCRIPTION OF THE INVENTION

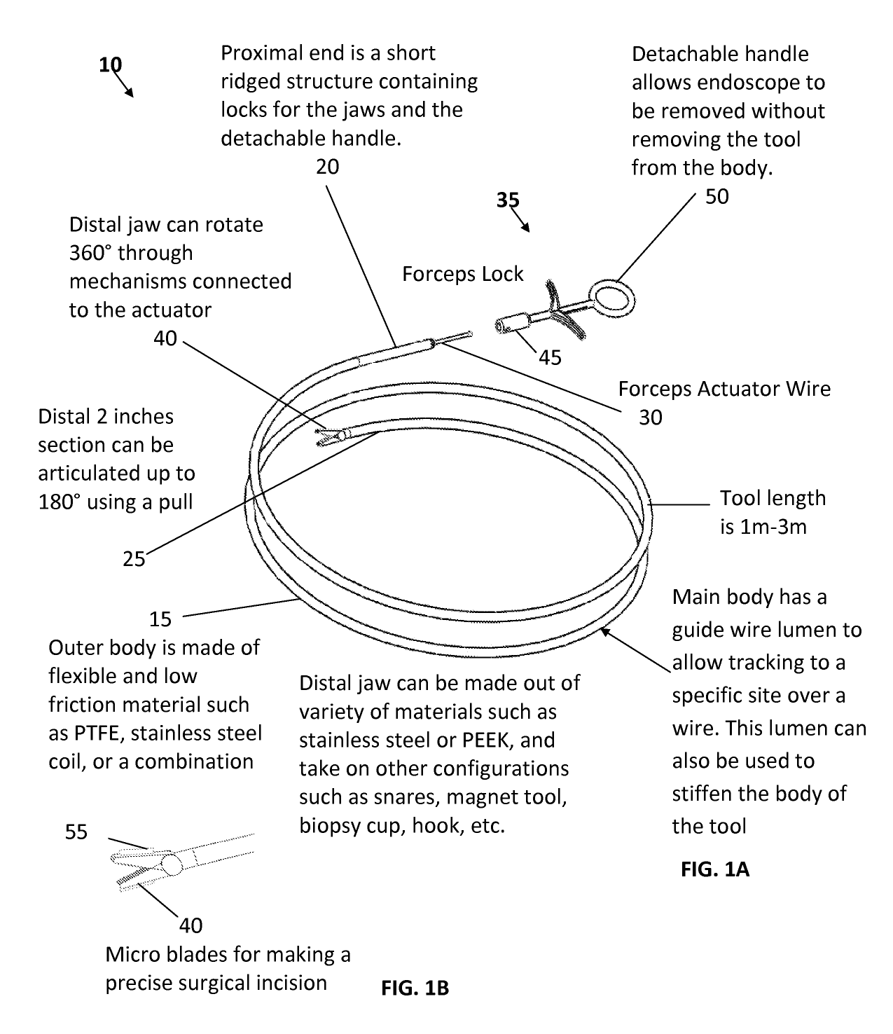

FIG. 1A shows one embodiment of a forceps system 10 that may be used in one or more of the methods disclosed below. The forceps system 10 includes a flexible body 15 with a proximal end 20 and a distal end 25. The flexible body 15 may be sized to fit within a tool channel or lumen of an endoscope. The flexible body 15 may also include other lumens, such as a guide wire lumen to allow tracking to a specific site over a guide wire. The guide wire lumen may also be used to direct the tool to a surgical site without the use of an endoscope. An actuator 30 extends through the body 15 and is removeably coupleable to a handle 35 near the proximal end 20 and an actuatable jaw 40 near the distal end 25. The actuatable jaw 40 may be a pair of opposed jaws, and depending on their configuration, the forceps system may be biopsy forceps, grasping forceps, and hemostatic forceps. In some embodiment, the actuatable jaw 40 may be removed and replaced by other actuatable tools, such as snares, magnet tool, biopsy cup, hook, or other suitable tools.

The handle 35 includes an attachment portion 45 and a forceps actuator 50. The attachment portion 45 is removeably coupled to the distal end 20 of the body with, for example, a set screw through the attachment portion 45 engaging the distal end 20. The forceps actuator 50 is operatively engageable to the actuator 30. In one embodiment, the actuator 30 has screw threads on the end and the forceps actuator 50 engages the threads. When operatively engaged, the forceps actuator 50 is configured to control the actuatable jaws by moving the actuator in and out. In addition, the forceps actuator 50 may also be configured to rotate the actuatable jaw 40 up to 360 degrees by rotating the actuator 30. A pull wire (not shown) may also be included to articulate the distal end 25 of the body along with the actuatable jaw 40. In use, when the forceps system 10 is within a tool channel of an endoscope and the handle 35 is uncoupled from the body 15, the endoscope can be withdrawn without removing the forceps system 10. The forceps jaw 40 may be locked with a forceps lock prior to removal of the handle. The forceps lock may be a set screw through the body 15 that engages and locks the actuator 30 in place. Locking the actuator 30 may also lock the actuatable jaw 40 in a fixed position. The forceps lock may also serve the purpose of an RF connection to the actuatable jaw (and other actuatable tools).

FIG. 1B shows an alternative embodiment for the actuatable jaw 40. In this embodiment, the actuatable jaw comprises one or more microblades 55 to create precise incisions in both endoscopic and NOTES surgery. The attached microblades allow better control over the size of the incision and thus permit easier fitting of correspondingly-sized balloon closure devices. The micro blades are mechanically limited and can help prevent uncontrolled incisions that other types of cutting devices may not, such as an RF needle knife.

The body 15 is made of a flexible and low friction material, such as PTFE, stainless coil, or a combination of both. The body 15 and actuatable jaw 40 (and other actuatable tools) are sized to be compatible with a 2.8 mm tool channel on an endoscope. The length of the forceps may be between 1 and 3 meters.

In many of the embodiments, the forceps system 10 may be used for general peritoneal exploration and tissue resection using NOTES approach with a flexible endoscope to perform a procedure, as will be described in more detail below. The endoscope may also include steering mechanisms that are used to steer the distal portion of the endoscope. The endoscope may include one or more tool channels that extend through the endoscope and provide an opening through which surgical instruments, such as the forceps system 10, may be inserted.

In one embodiment, a method of using a forceps system 10 includes advancing an endoscope into an internal surgical site, advancing jaws 40 and a first end 25 of an elongate flexible body 10 of the forceps system through a tool channel of the endoscope into the site, grasping a tissue with the forceps, removing a proximal handle 35 from a second end 20 of the elongate body 15 of the forceps, retracting the endoscope out of the site over the elongate body while the forceps grasps the tissue, and replacing the handle 35 on the forceps while the forceps grasps the tissue.

Other instruments may also be advanced through the endoscope tool channel, such as an RF catheter, to create a port or transgastric tract from the stomach into the peritoneal cavity. The flexible endoscope may be of the type that is typically used by gastroenterologists in treating the upper gastrointestinal tract and in accessing the esophagus or stomach. The endoscope allows the physician to visualize while performing procedures. The flexible endoscope may use fiber optics or a charge coupled device (CCD) mounted at the distal end of the endoscope to generate images.

During procedures through the mouth, the patient may be given a numbing agent that helps to prevent gagging. The endoscope is then passed through the mouth, into the stomach and through the port into the peritoneal cavity.

The endoscope may be used in locating a desired tissue site in the stomach. A transgastric tract is created through the stomach wall at the desired tissue site. The transgastric tract may be made using a RF catheter, RF guide wire, an endoneedle, or other suitable instrument. The size of the transgastric tract depends on the size of the device to go through, and have a diameter from 0.014″ to 0.250″.

The method disclosed below is directed toward Natural Orifice Translumenal Endoscopic Surgery (NOTES) from within the stomach into the peritoneal cavity. In one example, the resection and removal of the appendix using NOTES. In another example, the removal of a gallbladder using NOTES. The disclosed methods are shown as examples, as other combinations of devices may be combined to accomplish the same outcome.

### Example 1

### Resection of Appendix Using NOTES

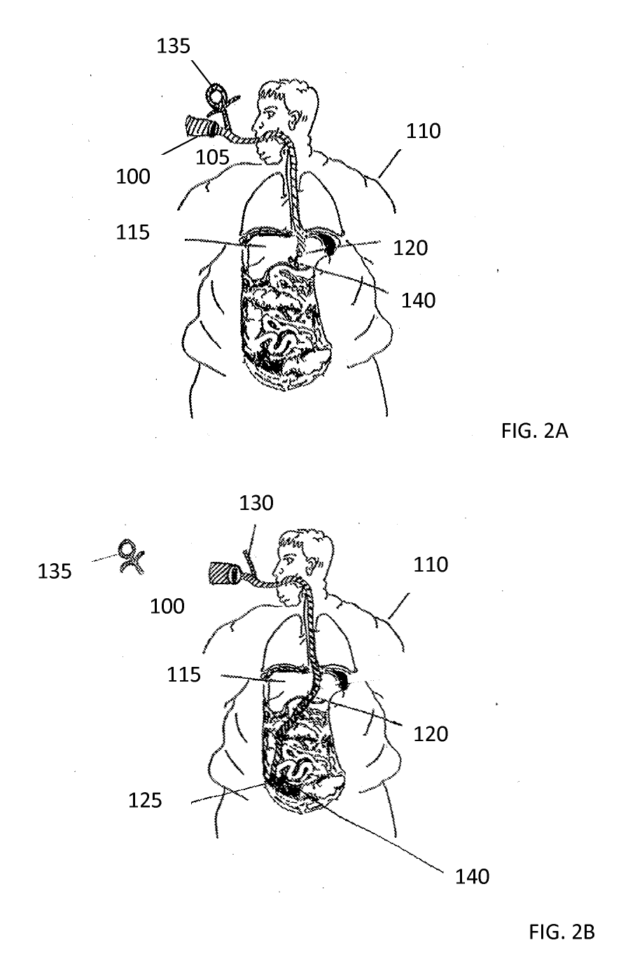

FIGS. 2A-2D show one embodiment using Natural Orifice Translumenal Endoscopic Surgery (NOTES) through the stomach to remove an appendix. Some of the equipment that may be used in this embodiment includes an endoscope, RF catheter, guide wire, forceps with removable handle, fluoroscope, endoscopic snare with electrocautery connection, electrocautery machine coupled to the snare, endoscopic grasper and closure devices.

One embodiment of the method includes the following steps:
1. Placing an endoscope 100 into the mouth 105 of a patient 110 until it is inside the stomach lumen 115.
2. Locating and creating a first transgastric tract or port 120 with a RF-Balloon Translumenal Crossing Device and RF guide wire. Dilating the balloon to maximum pressure for at least 30 seconds and then crossing into the peritoneal cavity. Removing the RF guide wire and replacing with a 0.035″ guide wire across the stomach wall.
3. Pushing the endoscope 100 through the first transgastric tract 120 into the peritoneal cavity to an internal surgical site, in this case the appendix 125.
4. Inspecting the peritoneum to verify appendicitis using the endoscope 100.
5. Removing the RF-Balloon Translumenal Crossing Device and tracking a forceps system 130 with removable handle 135 over the guide wire and grasping the appendix 125 with actuatable jaw 140 at the base near the colon. Locking the actuatable jaw 140 of forceps system 130, removing the handle 135 from the forceps and retracting the endoscope 100 out of the mouth, leaving the forceps system 130 in place. Replacing the handle 135 on to the forceps system 130 (FIG. 2C).
6. Replacing the endoscope 100 back into the lumen of the stomach and determining a second site such that it facilitates resection of the appendix. Dilating a second transgastric tract or port 145 using the RF-Balloon Translumenal Crossing Device on the stomach wall.
7. Placing endoclips around the base of the appendix to seal prior to resection.
8. Placing an endoscopic snare with electrocautery connection 150 through the endoscope tool channel and around the appendix 125. Begin resection using an electrocautery machine coupled to the snare. The actuatable jaw 140 of forceps system 130 may be manipulated to assist in placing the snare around the appendix.
9. Placing an endoscopic grasper in a second endoscope tool channel and grasping the appendix prior to completing the resection. Removing the appendix through the second transgastric tract or port created for the endoscope. The removal of the appendix may be done while removing the endoscope.
10. Inspecting for bleeding and leaks.
11. Closing the two transgastric tracts 120, 145 using appropriate means.

### Example 2

### Endoscopic Gallbladder Removal Using NOTES

One embodiment of the method includes the following steps:
1. Place an endoscope into the stomach and dilate a tract using the RF-Balloon Translumenal Crossing Device at an appropriate location on the stomach wall. Remove the RF wire from the balloon and place a guide wire across the dilated site.
2. After dilating, cross into the peritoneal cavity with the endoscope.
3. Inspect the peritoneum and verify the location of the gallbladder. Remove the balloon and track the handleless grasper with the right angle clamp tip over the wire and clamp the cystic duct.
4. Remove the handle from the endoscopic clamp tool and remove the endoscope. Reattach the handle.
5. Place the endoscope back into the lumen of the stomach and determine a working port for the endoscope such that it facilitates resection and removal of the gallbladder. Dilate a large tract using the RF-Balloon Translumenal Crossing Device and place a guide wire across the transgastric tract. Remove the balloon crossing device.
6. Attach endoclips to the cystic duct away from the common bile duct.
7. Place grasper through the endoscope. Using the grasper to manipulate the cystic duct, apply RF energy to the endoscopic clamp tool placed initially and dissect the cystic duct.
8. Release the endoscopic clamp tool and use the grasper tool in the endoscope to direct the clamp tool around the cystic artery. Remove the grasper.
9. Using a similar technique place endoscopic clips around the cystic artery.
10. Once the clips are attached replace the endoscopic grasper into the tool channel to manipulate and resects the cystic artery by applying RF energy to the endoscopic clamp.
11. Place an endoscopic RF tool with hook tip into the tool channel and dissect the gallbladder off the liver bed.
12. Remove the gallbladder carefully through the working port.
13. Close the initial transgastric site and the working port using appropriate means.

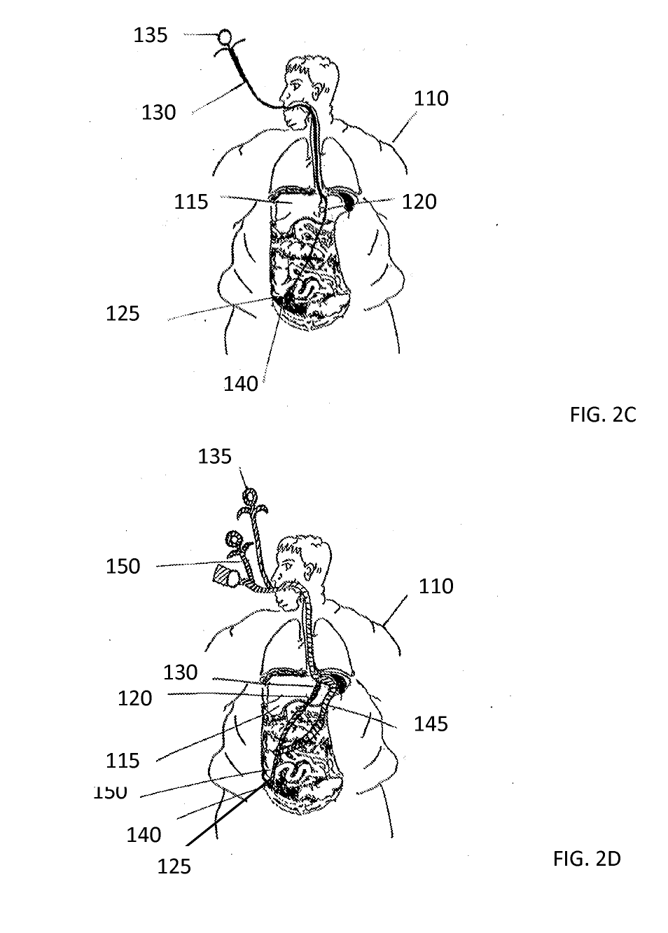

FIGS. 3A-3B show one embodiment of a closure device 200 compatible for use with natural orifice translumenal endoscopic surgery (NOTES) to close a transgastric tract or port once the surgery or procedure is complete. The closure device 200 includes a catheter 205 with a flexible body 210 having an inflation lumen 215. A balloon 220 is removeably coupled to the distal end of the catheter 205, the balloon 220 having a valve 225 in fluid communication with the inflation lumen 215. An inflation device 230 is in fluid communication with the inflation lumen 215 to inflate the balloon 220. The valve 225 may be designed to close and seal once it is disconnected from the inflation lumen 215. The catheter 205 and balloon 220 may have an antibiotic coating. The balloon 220 may be made from a compliant material, such as silicon or polyurethane. The closure device 200 may be sized to fit within a tool channel of an endoscope for delivery of the balloon to the tract or port. As shown in FIG. 3B, once in place across the transgastric tract, balloon 220 is inflated to close the tract 250; the catheter 205 is then uncoupled and removed, leaving the inflated balloon 220 in place.

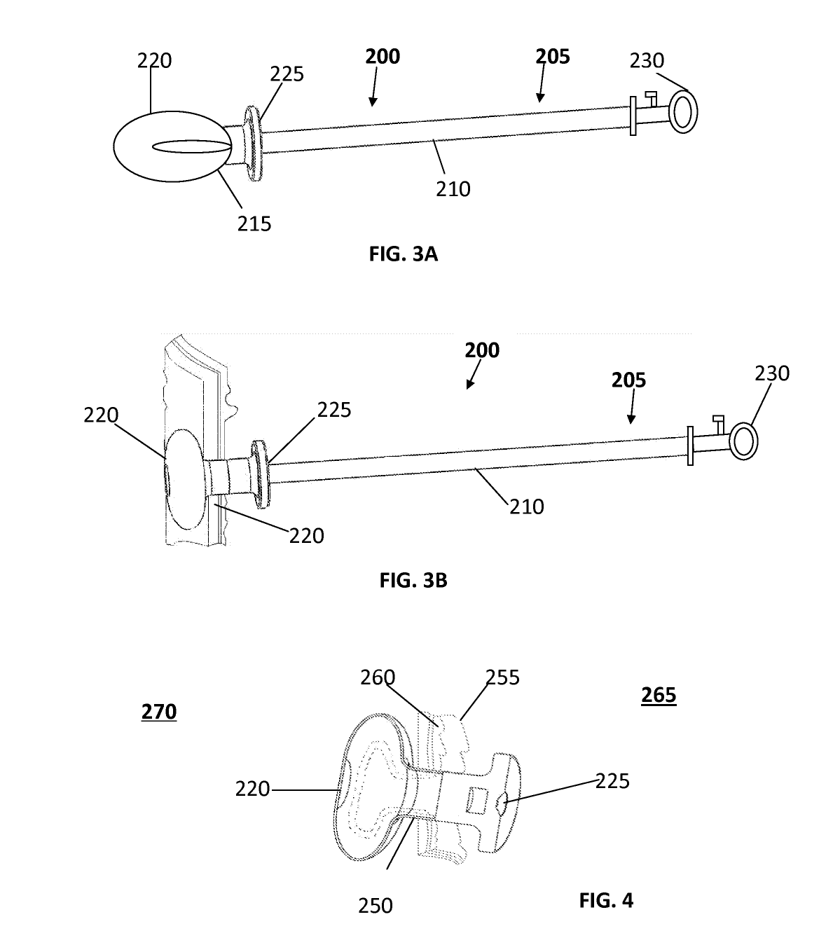

In one embodiment shown in FIG. 4, balloon 220 may be used as a temporary closure device to close a transgastric tract or port 250 created in the mucosa 255 and stomach wall 260 between the stomach 265 and peritoneum 270. The balloon 220 is delivered to the tract 250 on catheter 205. The delivery may be done through a tool channel of an endoscope. As the mucosa 255 and stomach wall 260 healing progresses, the fibers tighten and tract opening becomes smaller, and the balloon 220 changes shape (dotted lines). Once the healing is complete, the balloon 220 may be deflated and removed.

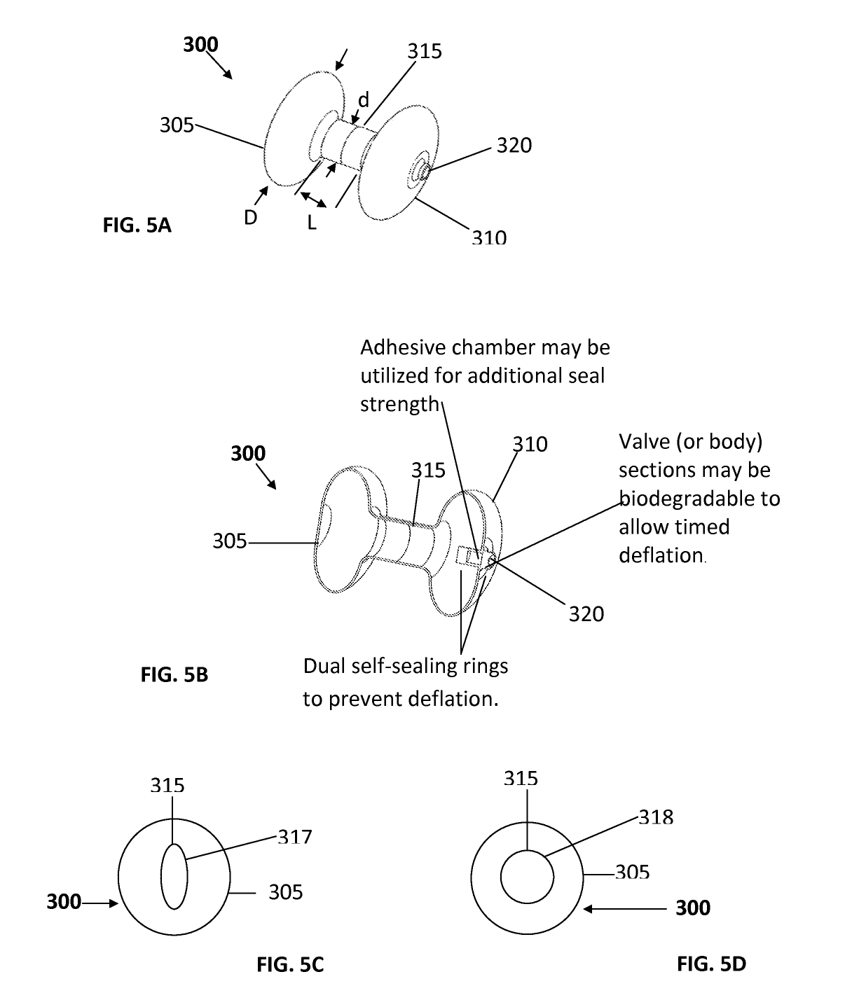

FIGS. 5A, 5B, 5C, and 5D show embodiments of a double balloon closure device 300 having a peritoneal side balloon 305 and a gastric lumen side balloon 310 joined by a narrow inflatable portion 315. The balloon closure device 300 is sized to close a transgastric tract or port, such as tract 250 discussed above. The narrow inflatable portion 315 may have a diameter between 5 mm to 50 mm and a length between 1 mm and 24 mm. The narrow inflatable portion 315 may be shaped to follow the general shape of the incision; thus the cross sectional shape of the narrow portion 315 may be circular, as shown in FIG. 5D or ovoid (or otherwise elongate) as shown in FIG. 5C. The diameters of balloons 305 and 310 are greater than the diameter of the narrow inflatable portion 315. Device 300 also includes an inflation valve 320 for inflating the balloons. The inflation valve may have dual self-sealing rings separated by an adhesive chamber. The components of the closure device 300, such as the valve 320 and/or body portions 305, 310, 315, may be biodegradable to allow timed deflation.

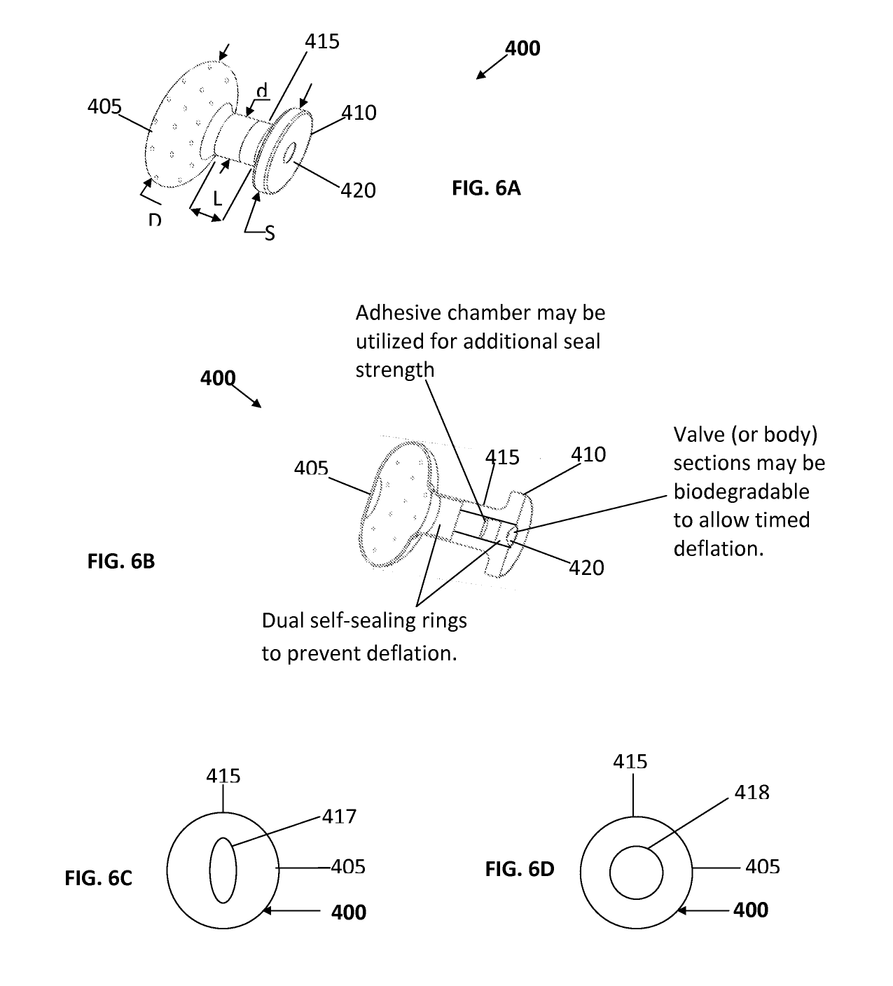

FIGS. 6A, 6B, 6C and 6D show embodiments of a balloon closure device 400 having a peritoneal side balloon 405 and a gastric lumen side disk 410 joined by a narrow inflatable portion 415 coupled to an inflation valve 420 for inflating the balloons. The balloon closure device 400 is sized to close a transgastric tract or port, such as tract 250 discussed above. The narrow inflatable portion 415 may have a diameter between 5 m to 50 mm and a length between 1 mm and 24 mm. The narrow inflatable portion 415 may be shaped to follow the general shape of the incision; thus the cross sectional shape of the narrow portion 315 may be circular, as shown in FIG. 6D or ovoid (or otherwise elongate) as shown in FIG. 6C. The diameter of balloon 405 and/or disk 410 is greater than the diameter of the narrow inflatable portion 415. The inflation valve may have dual self-sealing rings separated by an adhesive chamber. The components of the closure device 400, such as the valve 420 and/or body portions 405, 410, 415, may be biodegradable to allow timed deflation.

In some embodiments, the single balloon or dual balloon closure devices may contain one or more structures to dispense medication, bio glue or fibrin type sealant to promote or accelerate healing. FIG. 6E shows a dual balloon closure 428 with a structure 430 that delivers medication, bio glue or fibrin type sealant, or biomaterial plug. Structure 430 communicates with the delivery catheter through a lumen of the structure that connects the valve on the balloon to the proximal port on the delivery catheter. Medication (for example, antibiotics or other types of medication that increase the healing process), bio glue or fibrin type sealant may be injected into the structure through the proximal port to dispense sealant around the incision and over the balloon in the peritoneal cavity.

As shown in FIG. 6F for a single balloon closure device, in other alternative embodiments, the single balloon or dual balloon closure devices 435 may comprise concentric balloons on the peritoneal side. The inner balloon 440 may communicate through a lumen 470 with an inflation port 460 to form an anchor on the peritoneal side. The outer balloon 450 may be perforated and comprise a second channel in communication with the delivery catheter to dispense medicine, bio glue or sealant through the perforations so as to fill some or all of the remaining gaps in between the closure device and the incision site through the stomach wall and to effectively seal and/or cover up the incision site.

As shown in FIG. 6G for a single balloon closing device, in other alternative embodiments, the single or double balloon closure devices on the peritoneal side comprise anchoring balloons that are perforated 430. The perforated balloons desirably contain a type of biocompatible sealant, capable of solidification within a short amount of time. Thus, a biocompatible type of sealant, such as fibrin, may be both be dispersed over the incision site and used to keep the anchor inflated on the peritoneal side once it solidifies.

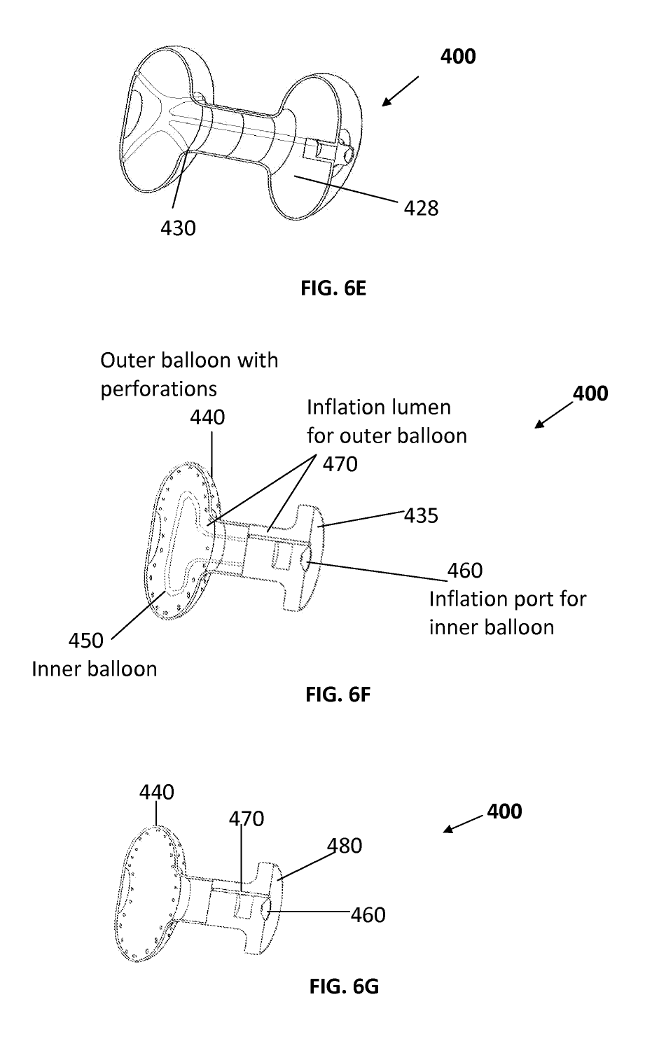

FIG. 7 shows one embodiment of a closure device catheter 500 for use with the balloon closure devices. The catheter 500 includes a flexible shaft 505 having a proximal end 510 and a distal end 515. The shaft 505 may be compatible with a gastric endoscopic tool channel. An inflation lumen 520 extends through the shaft 505 and is coupled to an inflation port 525 on the proximal end. The distal end 515 is configured to engage a valve of the balloon to inflate it. In some embodiments, the distal end 515 is configured to pierce through a closure balloon device to inflate the balloon and then seal the piercing using an optional adhesive dispensing lumen 530 coupled to an adhesive lumen port 535. The catheter 500 may also have a guide wire port 540 and guide wire lumen 545 for tracking the catheter 500 over a guide wire.

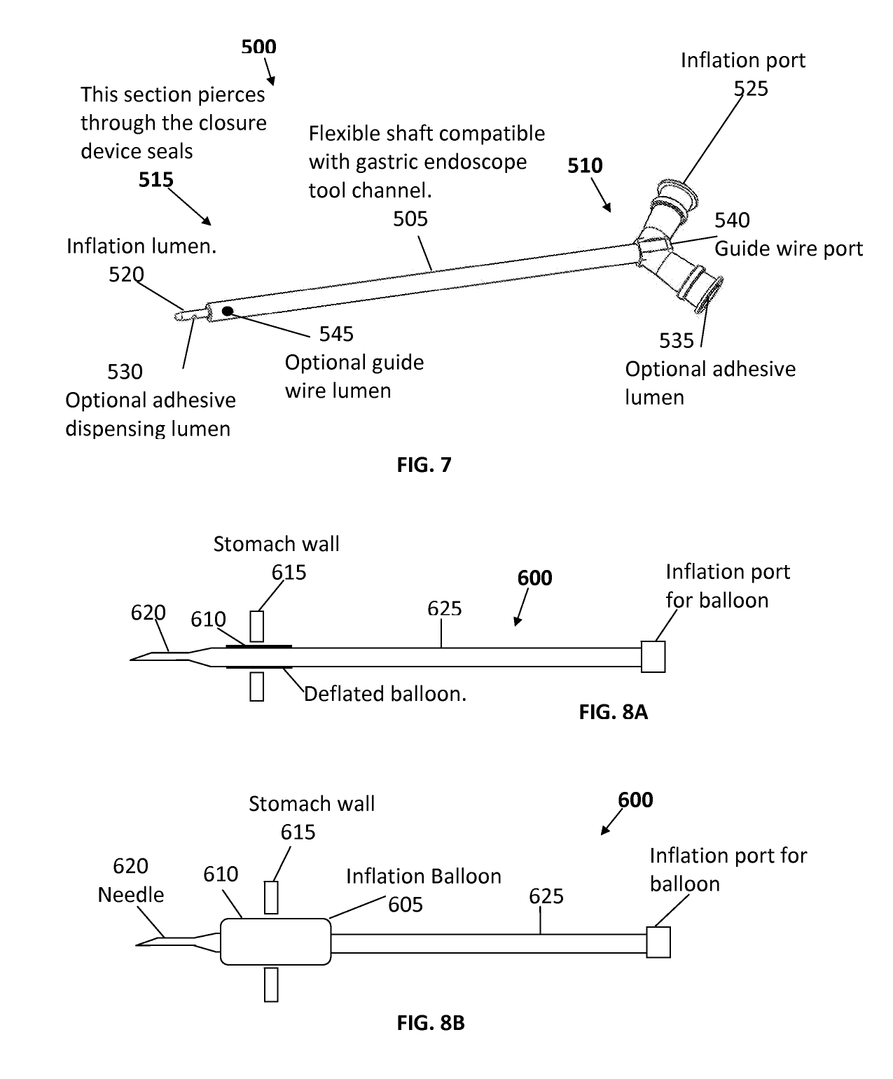

FIG. 8A shows one embodiment of a Balloon Translumenal Crossing Device 600 having a balloon 605 on a distal end for creating and dilating a transgastric cut 610 in a stomach wall 615 made by a fixed needle 620 at the tip of the catheter body 630 to facilitate initial incision prior to dilation. It is to be noted that Balloon Translumenal Crossing Device 600 may optionally be equipped with a removable RF wire 622 or an electrocautery blade (not shown) within a guide wire lumen 625 incorporated within the catheter body 630 for the purpose of creating and dilating a transgastric cut 610. As shown in FIG. 8B, the balloon 605 may be inflated with enough pressure to dilate the transgastric cut 610 opening in the stomach wall 615, creating a working port or transgastric tract. In other embodiments, the balloon 605 may be inflated to create space within the peritoneal cavity 635. The balloon 605 may be formed of either a compliant or non-compliant material such as, e.g., polyurethane, polyethylene, polyester or a rubber material such as silicone, depending on the use of the catheter. Once the RF wire 622, needle 620, or electrocautery blade is removed, the lumen 625 may be used to place a guide wire across the transgastric cut 610. A handle 640 on the proximal end of the catheter 600 may also removable to allow removal of an endoscope without removing the catheter. The catheter 600 may also serve as a guide rail for an endoscope or any other tool with an appropriate lumen. As shown in FIG. 8C, the balloon may comprise microblades 605 that cut stomach muscle tissue and thus minimize the tearing of the muscle tissue.

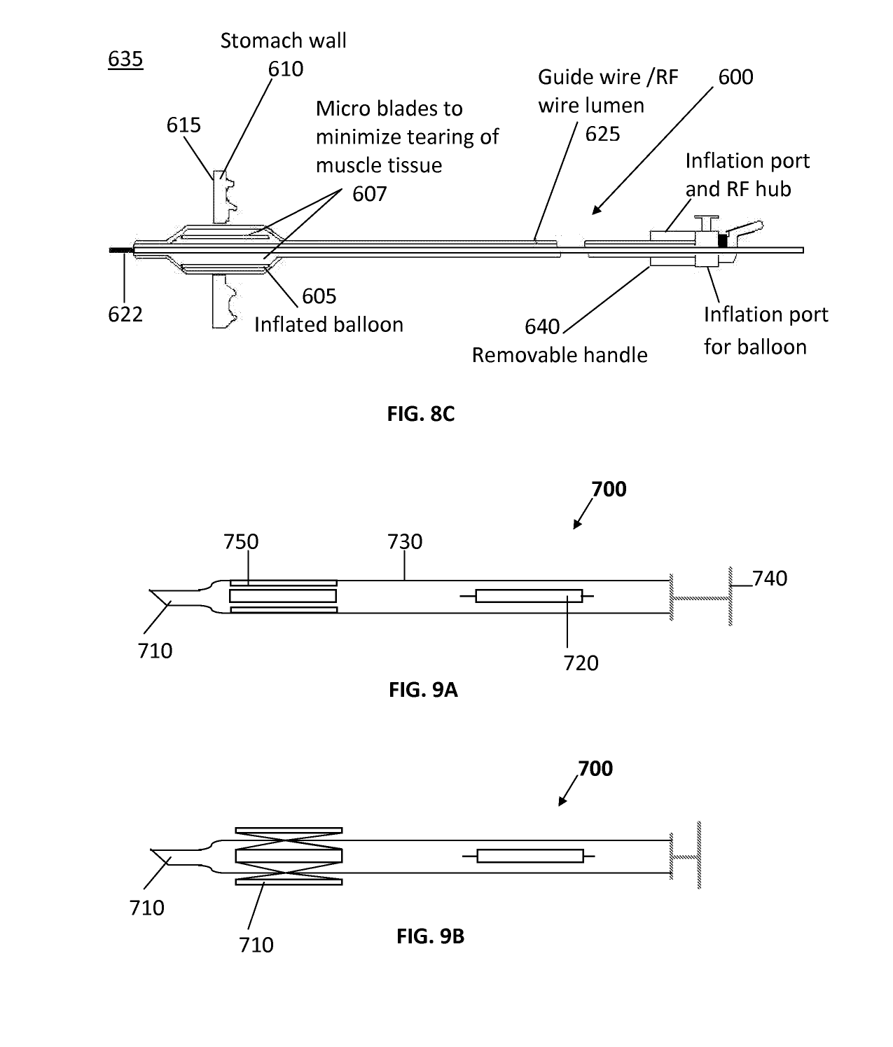

FIG. 9A shows an embodiment of a Mechanical Translumenal Crossing Device 700 having deployable dilator arms that dilate the transgastric cut 710 opening in the stomach wall. A spring-loaded laparoscopical surgery knife 710 or other tissue penetrating structure is released by a trigger 720 located on the handle 730. Once the initial cut or opening is formed or finished, and a distal end of the crossing device is advanced into the initial opening, a plunger 740 is used to deploy the dilator arms 750 to dilate the transgastric cut. FIG. 9B shows the Mechanical Translumenal Crossing Device with its dilator arms deployed. Advantageously, the elongate bodies or catheters of the tissue penetrating and dilating structures may be sufficiently flexible for transgastric (and other NOTES) procedures.

Although the foregoing invention has been described in some detail by way of illustration and example, for purposes of clarity of understanding, it will be obvious that various alternatives, modifications and equivalents may be used and the above description should not be taken as limiting in scope of the invention which is defined by the appended claims.

What is claimed is:

1. A method comprising:
advancing an endoscope into an internal surgical site;
advancing a first end of an elongate flexible body of a forceps through a tool channel of the endoscope into the site;
grasping a tissue with the first end of the forceps;
removing a proximal handle from a second end of the elongate body of the forceps;
retracting the endoscope out of the site over the elongate body while the forceps grasps the tissue;
replacing the handle on the forceps while the forceps grasps the tissue.

2. The method of claim 1, wherein the first end of the forceps includes actuatable jaws removeably coupled to the first end.

3. The method of claim 2, wherein the actuatable jaws may be removed and replaced by another actuatable device, including at least one of: a snare, a magnetic tool, a biopsy cup, a hook, or other suitable actuatable device.

4. A forceps system for use with an endoscope, comprising:
an elongated body extending from a proximal end and a distal end having one or more internal lumens;
an actuator slidably positioned within a first lumen;
an actuatable device removeably coupled to a first end of the actuator near the distal end; and
a handle removeably coupleable to the proximal end of the body, the removable handle having a forceps actuator operatively engageable with a second end of the actuator so as to control the actuatable device when the handle is coupled to the body.

5. The forceps of claim 4, wherein the forceps actuator is configured to move the actuatable device from a first position to a second position.

6. The forceps of claim 4, wherein the forceps actuator is configured to rotate the actuatable device.

7. The forceps of claim 4, wherein the actuatable device includes at least one of: actuatable jaws, a snare, magnetic tool, a biopsy cup, a hook, or other suitable actuatable device.

8. The forceps of claim 4, wherein the actuatable device may be removed and replaced by another actuatable device.

9. The forceps of claim 4, wherein the actuatable device may be connected to an RF generator via the actuator cable.

10. The forceps of claim 4, further comprising an actuator wire locking mechanism configured to lock the actuator wire within the first lumen.

11. The forceps of claim 10, wherein locking the actuator wire also locks the actuatable device.

12. The forceps of claim 4, wherein one of the lumens is a guide wire lumen.

13. The forceps of claim 4, further comprising a pull wire within the body configured to articulate the distal end including actuatable device.

14. The forceps of claim 4, wherein the body and actuatable device are configured to slide within a tool or accessory channel of the endoscope.

15. The forceps of claim 4, wherein the actuatable device comprises jaws movably supporting one or more blades.

16. A method of using forceps with a removable handle in natural orifice translumenal endoscopic surgery (NOTES), the method comprising:
creating a first port from a patient's stomach into a peritoneal cavity;
advancing an endoscope orally into the stomach, through the first port to an internal surgical site;
advancing a first end of a forceps through a tool channel of the endoscope to the surgical site;
engaging a tissue at the surgical site with the first end of the forceps;
removing the handle from the forceps;
retracting the endoscope out of the mouth, leaving the forceps in place;
replacing the handle on the forceps;
creating a second port from the stomach into a peritoneal cavity;
advancing the endoscope orally into the stomach, through the second port into the peritoneal cavity to the internal surgical site;
advancing a first end of a tool through the tool channel of the endoscope to the surgical site;
manipulating the tissue at the internal surgical site with the forceps; and
performing a procedure at the surgical site with the tool and forceps.

17. The method of claim 16, wherein the first end of the forceps is an actuatable device and includes at least one of: actuatable jaws, a snare, magnetic tool, a biopsy cup, a hook, at least one blade or other suitable actuatable device.

18. A balloon closure device for temporarily closing a transgastric tract comprising:
a delivery catheter having a proximal end and a distal end;
an inflation lumen and a guide wire lumen within the catheter; and
an inflatable closure balloon removeably coupled to the distal end, the balloon having a pressure valve in fluid communication with the inflation lumen such that the balloon remains inflated once uncoupled from the catheter, the balloon being sized to temporarily close the transgastric tract when inflated.

19. The device of claim 18, wherein the balloon has an antibiotic coating.

20. The device of claim 18, wherein the balloon comprises a compliant material that allows it to conform to a smaller size as the transgastric tract closes.

21. The device of claim 18, wherein the balloon comprises silicon or polyurethane or a biodegradable polymer.

22. The device of claim 18, wherein the balloon closure device is designed to be rejected into the gastric lumen after the healing is complete.

23. The device of claim 18 wherein the inflatable balloon closure comprises a narrow inflatable portion having an elongate cross-section so as to sufficiently fill a shape of a transgastric cut to provide a tight closure during the healing.

24. The device of claim 18 wherein the inflatable balloon closure comprises a peritoneal side with perforations, and further comprising a biocompatible sealant capable of solidification within less than 30 minutes of application when the peritoneal side is inflated therewith, wherein the biocompatible sealant keeps the peritoneal end inflated once the biocompatible sealant solidifies.

25. The device of claim 18 wherein the inflatable balloon comprises a peritoneal side and a mucosal side, the peritoneal side comprising an inner inflatable balloon portion and an outer perforated balloon portion having perforations, and further comprising a biocompatible sealant capable of solidification within less than 30 minutes of inflation of the balloon therewith, wherein the biocompatible sealant keeps the peritoneal end inflated once the biocompatible sealant solidifies.

26. The device of claim 18 wherein the inflatable balloon closure is contains or comprises medication so as to release the medication into the transgastric tract to speed up the healing when in use.

27. A method of closing a transgastric tract, the method comprising:
advancing a closure device delivery catheter to the transgastric tract by means of a guide wire or endoscope;
positioning an inflatable balloon closure device on a first end of the closure device across the transgastric tract; and
inflating the balloon to seal the transgastric tract.

28. The method of claim 27, further comprising uncoupling the inflated balloon from the delivery catheter and withdrawing the delivery catheter.

29. The method of claim 27, wherein advancing the closure device to the transgastric tract comprises positioning an endoscope proximate the transgastric tract and advancing the closure device through a tool channel of the endoscope.

30. The method of claim 27, further comprising removing the balloon once the transgastric tract has healed.

31. The method of claim 27, wherein the balloon closure device is rejected into the gastric lumen after the healing is complete.

32. A transluminal crossing device comprising:
an elongated flexible body extending from a proximal end to a distal end;
a tissue penetrating tip disposed at the distal end so as to form a penetration of a wall of a body lumen; and
an expandable structure disposed proximally of the tip, the expandable structure having a small-profile configuration suitable for advancement of the expandable structure into the penetration and a large-profile configuration, expansion of the expandable structure from the small-profile configuration to the large profile configuration suitable for expanding the penetration when the wall surrounds the expandable structure.

33. The transluminal crossing device of claim 32, wherein the radially expandable structure comprises a mechanism, a plurality of arms of the mechanism deploying radially from along the body when the expandable structure expands from the small-profile configuration to the large-profile configuration.
